(19) United States
(12) Patent Application Publication
(10) Pub. No.: US 2026/0308200 A1
(43) Pub. Date: Oct. 8, 2026

(54) NATIVE PEPTIDE CYCLIZATION, SEQUENTIAL CHEMOSELECTIVE AMIDATION IN WATER

(71) Applicant: The Research Foundation for the State University of New York, Albany, NY (US)

(72) Inventors: Qiang Zhang, Albany, NY (US); Huan Chen, Albany, NY (US)

(21) Appl. No.: 19/481,821

(22) PCT Filed: May 6, 2024
(86) PCT No.: PCT/US24/28017

§ 371 Date: Nov. 5, 2025

Related U.S. Application Data
(60) Provisional application No. 63/464,375, filed on May 5, 2023.

(51) Int. Cl. C07K 1/113; C07K 1/02

(52) U.S. Cl. CPC C07K 1/113; C07K 1/026

(57) ABSTRACT

Disclosed herein are processes directed to Native Peptide Cyclization (NPC) and chemoselective methods enabling intramolecular and intermolecular peptidyl ligation without the need for premodification of starting peptides. NPC cyclizes unprotected linear peptides through controlled, sequential C- and N-terminal NPC simplifies peptide ligation, easing the labor-intensive nature of peptide synthesis, efficient cyclic peptide preparation and enabling cost-effective macrocycle-based therapeutics.

### CROSS-REFERENCE TO RELATED APPLICATIONS

This application claims the benefit of U.S. Provisional Application No. 63/464,375, filed May 5, 2023, the contents of each of which are hereby incorporated by reference into the application.

### STATEMENT REGARDING FEDERALLY SPONSORED RESEARCH

This invention was made with government support under grant number GM138336, awarded by the National Institute of Health. The government has certain rights in the invention.

### FIELD OF THE INVENTION

The present disclosure is generally in the field of organic chemistry. More particularly, it concerns methods of making peptides, polypeptides, protein, and the like. In embodiments, the disclosure relates to methods of making cyclized peptide, and elongated peptides and proteins.

### BACKGROUND OF THE INVENTION

The extensive functionalities exhibited by peptides and proteins in essential life processes underscore the importance of their chemical customization and preparation. Biomedically, they hold crucial significance as therapeutic agents and biochemical tools, as evidenced by the recently approved therapeutic peptides and proteins by FDA. Despite significant advancements in polypeptide synthesis protocols, the extant methods rely on two fundamental principles for linking polypeptides: premodifying terminal residues (e.g., —COOH to —COSR) and side chain protective groups (e.g., -tBu). These prerequisites introduce additional steps for incorporating artificial residues at ligation sites or protective groups, resulting in an intricate and labor-intensive process. Furthermore, linear proteins or peptides produced via recombinant protein technology typically remain unmodified, hindering the biologically mediated formation of cyclic peptides through covalent amide bond connections. Connecting unmodified peptidyl N- and C-termini directly at the ligation site faces a major challenge due to the lack of a chemo-selective chemical method accommodating all 20 proteinogenic amino acids.

As depicted in FIG. 1a, the differentiation of unprotected, proteinogenic peptides generated chemically or biologically poses a significant hurdle, and the ligation of linear peptides, through either intermolecular (elongated peptide) or intramolecular methods (cyclized peptide), remains unattainable chemically. Challenges include discerning reactivity variations among diverse proteinogenic amino acid side chains and maintaining original stereochemistry at the ligation site. Additionally, unprotected peptides' intrinsic polarity limits their solubility to many solvents, often favoring aqueous solutions. Nevertheless, utilizing aqueous solutions hinders peptide ligation by inhibiting the necessary formation of water, a byproduct of the peptidyl amidation equilibrium. Thus, new efficient and economical processes for making cyclized peptides is needed for new therapeutic agents and biochemical tools.

### BRIEF SUMMARY OF THE INVENTION

In embodiments, the present disclosure provides a new sequential chemoselective activation method to achieve intramolecular and intermolecular peptide ligation without reliance on residue premodification or orthogonal protection. All the linear polypeptides and proteins included in this disclosure can be obtained from protein expression, chemical solid phase synthesis, and commercially available sources. In embodiments, the present disclosure provides new methods of making cyclized peptides, as more fully disclosed herein below.

Embodiments disclosed herein, are directed to a method of cyclizing peptides. The method having the steps of: i) reacting a at least one unprotected peptide containing a plurality of amino acids in an acidic aqueous solution, said peptide having a C-terminus end and an N-terminus end; ii) activating the C-terminus of at least one amino acid with an electrophilic activator in the solution; and iii) maintaining the solution at a temperature of about 0° C. to about 99° C. to form at least one stable amide bond that provides an N-to-S shift and at least one cyclized peptide. According to an embodiment, the method of cyclizing peptides, the N-terminal amino acid has a thiol group, and according to another embodiment the N-terminal is a cysteine or selenocysteine amino acid. According to an embodiment, the acidic aqueous solution contains hydrochloric acid, trifluoroacetic acid, or acetic acid to provide a pH of about 2 to about 5. According to another embodiment, the at least one peptide is a linear peptide. According to another embodiment, the electrophilic activator is at least one water-soluble isonitrile. According to yet another embodiment, the isonitrile is at least one selected from the group consisting of isopropyl isonitrile, tert-butyl isonitrile, acetonitrile, and methyl isonitrile. According to another embodiment, the peptide is an HPLC-purified linear peptide. According to another embodiment, the cyclized peptide contains from about 5 to about 20 amino acid residues, and the peptide is obtained from chemical solid phase synthesis or protein expression.

Embodiments disclosed herein, are directed to a method of making cyclized peptides, wherein the steps of the method are directed to: reacting at least one unprotected peptide containing a plurality of amino acids in a buffered aqueous solution having a first pH of at least about 7 to about 12; activating an N-terminus of at least one amino acid with an electrophilic activator in the solution at the first pH; reducing the first pH of the solution to a second pH that is from about 2 to about 5 and maintaining temperature of the solution at the second pH from about 30° C. to about 50° C. to provide at least one cyclized peptide. Further embodiments are directed to the N-terminus amino acid containing a thiol group, or the N-terminus is a cysteine or selenocystine amino acid. Still further embodiments are directed to a buffered aqueous solution is a phosphate-buffer solution (PBS) and the first pH is about 7.8 and the second pH is 4.7.

Other embodiments disclosed herein are directed to a method of making cyclized peptides, wherein the method provides steps of: dissolving at least one unprotected peptide salt containing a plurality of amino acids in an aqueous solution at a temperature of about 20° C. to about 30° C. to provide an acidic solution and least one cyclized peptide; and, wherein the peptide contains N-terminus cysteine amino acid.

Still further embodiments disclosed herein, are directed to a method of elongating peptides/proteins. The steps of the method are directed to: i) reacting at least one unprotected first peptide containing a plurality of amino acids in an acidic aqueous solution having a pH of about 2 to about 5 at a temperature from about 0° C. to about 99° C., said first peptide having a C-terminus end and an N-terminus end and at least one thiol containing amino acid that is not the C-terminus or the N-terminus; ii) activating the C-terminus carboxylic acid of at least one amino acid of the first peptide with an electrophilic activator in the solution to provide thiolysis of the least one thiol containing amino acid to form at least one macro-thiolactone first peptide; iii) adding a at least one unprotected second peptides to the solution, said second peptide having a C-terminus end and an N-terminus end and at least one C-terminus containing a thiol group and at least one amino acid containing a thiol group that is not the C-terminus; and iv) increasing the pH of the acidic aqueous solution to about 6 to about 8 to provide for ligation of the at least one macro-thiolactone first peptide and at least one second peptide to form at least one elongated peptide.

The method of elongating peptides/proteins, further contains the steps of; reducing the solution comprising at least one elongated peptide to a pH of about 2 to about 5; adding an unprotected third peptide, the third peptide having a C-terminus end and an N-terminus end and at least one C-terminus containing a thiol group and at least one thiol containing amino acid that is not the C-terminus; and adjusting the solution pH to about 6 to about 8 and temperature to about 25° C. to provide for ligation of the elongated peptide and third peptide to produce an elongated protein. According to embodiments the amino acid containing a thiol group is a cysteine or selenocysteine amino acid. According to another embodiment of the method, the first, second, and third peptides are HPLC-purified linear peptides; the unprotected first, second, and third peptides are obtained from chemical solid phase synthesis or protein expression; and the unprotected first, second, and third peptides are peptide HPLC-purified linear peptide.

As such, chemical synthesis offers robust tactics for structural alterations of peptides and proteins. It remains a labor-intensive and complex process due to the challenges in selectively modifying diverse amino acid side chains and termini. Direct α-peptide ligation without premodification is a significant hurdle, especially when aiming to include all proteinogenic amino acids at the ligation site. Embodiments disclosed herein are directed to Native Peptide Cyclization (NPC) and chemoselective methods enabling intramolecular and intermolecular peptidyl ligation without the need for premodification of starting peptides. NPC cyclizes unprotected linear peptides through controlled, sequential C- and N-terminal activation via pH modification. Water-based NPC simplifies peptide ligation, easing the labor-intensive nature of peptide synthesis, efficient cyclic peptide preparation and enabling cost-effective macrocycle-based therapeutics.

### BRIEF DESCRIPTION OF THE DRAWING

FIG. 1a, FIG. 1b, and FIG. 1c, present the ligation of unprotected peptide side chains and termini in water.

FIG. 2 presents an evaluation of NPC for its selectivity and epimerization propensity utilizing NPC conditions, i.e., a) Isonitrile 2 (10 equiv), H2O (5 mM), 38° C., 8-48 h.; b) NaOH (1 M) for 10 min.; and c) in some cases, cyclized products may undergo S-alkylation with isonitrile at the cysteine thiol after NPC. The addition of 1MNaOH (10 min.) allows for the recovery of unmodified cyclopeptides.

FIG. 3. Presents Asp/Glu selectivity evaluations under NPC conditions to validate the connectivity of the cyclopeptide structures, a conventional cyclization method with appropriate side chain protection was employed (HATU/DIPEA/DMSO). Samples from different approaches were co-injected in HPLC to confirm the desired connectivity.

FIGS. 4a-4g presents a tentative NPC mechanism and NPC enables in situ elaborations. One-pot post-cyclization modification of cyclo-CWNYA FIG. 4a TCEP, GSH, VA-044, H2O, 38° C., (43%) 2-step. FIG. 4b BnBr, 1MNaOH, rt, 30 min, (71%) 2-step. FIG. 4c crude HPLC trace of the cyclization reaction for cyclo-CWNYA preparation (20 to 60% solvent B over 20 min). FIG. 4d crude reaction trace for one-pot benzylation (30 to 70% solvent B over 20 min). FIG. 4e multiple peptide cyclizations were performed in one-pot using NPC. FIG. 4f isopropyl isonitrile (10 equiv), H2O, 38° C., 12 h. FIG. 4f HPLC of rude cyclo-CWNYA and 21 revealed acceptable product purities. FIG. 4g the application of NPC to a mixture of linear peptides led to the selective generation of six distinct macrocycles derived from their corresponding linear precursors.

FIGS. 5A and 5B present a schematic of the structural characterizations of H-CWNYA-OH (S1), CWNYA S2), cyclo-CWNYA S3, and N-formate-CWNYA S4 in connection with NPC conditions presented in Table S1.

FIG. 6 present a schematic of the ligation of unprotected donor peptide and unprotected acceptor peptide in water.

FIG. 7 presents a schematic of the peptide/protein elongation method of the synthesis of snake toxin protein by elongation method)

### DEFINITIONS

As discussed herein, amino acids are the fundamental molecule that serves as the building block for proteins and contain both amino and carboxylic acid functional groups. Although over 500 amino acids exist in nature, the most important are the alpha-amino acids, from which proteins are composed. Only 22 alpha amino acids appear in the genetic code.

Amino acid residues occur when two or more amino acids combine to form a peptide, the elements of water are removed, and what remains of each amino acid is called an amino-acid residue. As such, amino acid residues are carbon chains that contain an amino group (NH3+), a carboxyl group (COOH), and a carbon side chain. Amino acids are linked by the carboxyl group on one amino acid-binding to the amino group on another amino acid in a bond known as a peptide bond.

As discussed herein, amino acids sidechains are the portion of alpha-amino acid structure other than the amino group (or the ammonium group in an ionic form), the alpha carbon, and the carboxylic acid group (or the carboxyl group in an ionic form).

As discussed herein, amino acids termini are located at the ends of a peptide, i.e., the end with a free amino group is called the N-terminal amino acid residue. The end with a free carboxyl group is called the C-terminal amino acid residue, and peptides are named from the N-terminal acid residue to the C-terminal amino acid.

As discussed herein, a polypeptide is a string of covalently bonded amino acids which are not folded into any specific structure, whereas a protein is a string of covalently bonded amino acids that has folded into its correct shape. A peptide is a short chain of amino acids (typically 2 to 50), a longer chain of linked amino acids (e.g., 51 or more) is a polypeptide, and a protein generally has at least 50 amino acid residues.

For purposes of the methods disclosed herein, the term peptide/protein is understood to be inclusive of any covalently linked canonical and noncanonical amino acid residues via amide bonds.

As used herein, chemoselectivity refers to the preferential reaction of a chemical reagent with one of two or more different functional groups.

As used herein, HPLC High-performance liquid chromatography is a technique in analytical chemistry used to separate, identify, and quantify each component in a mixture.

As discussed herein, cyclopeptide, also known as cyclic peptides, are polypeptides formed from amino acids arranged in a cyclic ring structure.

As discussed herein, kinetic control or kinetically controlled is a chemical reaction in which the product ratio is determined by the rate at which the products are formed. Also, a thermodynamic product would be the more stable product formed in a chemical reaction; and thermodynamic control is a reaction in which the product ratio is determined by the relative stability of the products.

As discussed herein, the N-to-S is driven by the formation of thermodynamic stable amide bonds, compared to breaking less thermodynamic thioester bonds. Thus, the N-to-S shift in the NPC reaction is triggered by the amide bond formation.

The Passerini reaction, as discussed herein, is a chemical reaction involving an isocyanide, an aldehyde (or ketone), and a carboxylic acid to form a α-acyloxy amide.

The Ugi multicomponent reaction, as discussed herein, is performed by mixing an aldehyde, an amine, a carboxylic acid, and an isocyanide and is among the most important isocyanide-based multicomponent reactions (MCRs), allowing multiple bond formations (C—C and C—N) in a single synthetic step.

As discussed herein, a nucleophile is a chemical species that forms bonds by donating an electron pair. All molecules and ions with a free pair of electrons or at least one pi bond can act as nucleophiles. Because nucleophiles donate electrons, they are Lewis bases.

As discussed herein, nucleophilicity refers to the ability of a nucleophile to displace a leaving group in a substitution reaction.

As discussed herein, peptides are categorized according to how many amino acids make up the chain, i.e., oligopeptides have few amino acids, while polypeptides are longer chains. Dipeptides, tripeptides, and tetrapeptides have two, three, and four amino acids, respectively. For purposes herein, the terms oligopeptides, peptides, and polypeptides are synonymous.

As discussed herein, macrocyclic peptides (peptidyl macrocycle) are predominantly peptide structures bearing one or more rings and spanning multiple amino acid residues.

As discussed herein, the abbreviation PG refers to a protecting group, which is a molecular framework that is introduced onto a specific functional group in a poly-functional molecule to block its reactivity under reaction conditions needed to make modifications elsewhere in the molecule.

For purposes herein, in contrast to unaltered peptides protected peptides include the protection of amino acid reactive functionalities including the α-amino group, the side chain (amines, carboxylic acids, alcohols, and thiols), or the carboxylic acid terminus is an essential strategy in peptide chemistry.

As discussed herein, orthogonal moieties relate to where one component of a multicomponent system reacts with a particular reagent under a specific set of conditions while the others do not, even though they are all present together in the same reaction vessel.

As discussed herein, solid-phase synthesis is a technique for peptide synthesis. Usually, peptides are synthesized from the carbonyl group side (C-terminus) to amino group side (N-terminus) of the amino acid chain in the SPPS method, although peptides are biologically synthesized in the opposite direction in cells.

As discussed herein, protein expression is the cellular process of synthesizing proteins from DNA, involving transcription to mRNA, translation to polypeptide chains, and post-translational modifications for functional protein formation. Protein expression starts the peptide sequence biological synthesis from N-terminus and ends at C-terminus.

Before embodiments are further described, it is to be understood that this disclosure is not limited to particular embodiments described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### DETAILED DESCRIPTION OF THE INVENTION

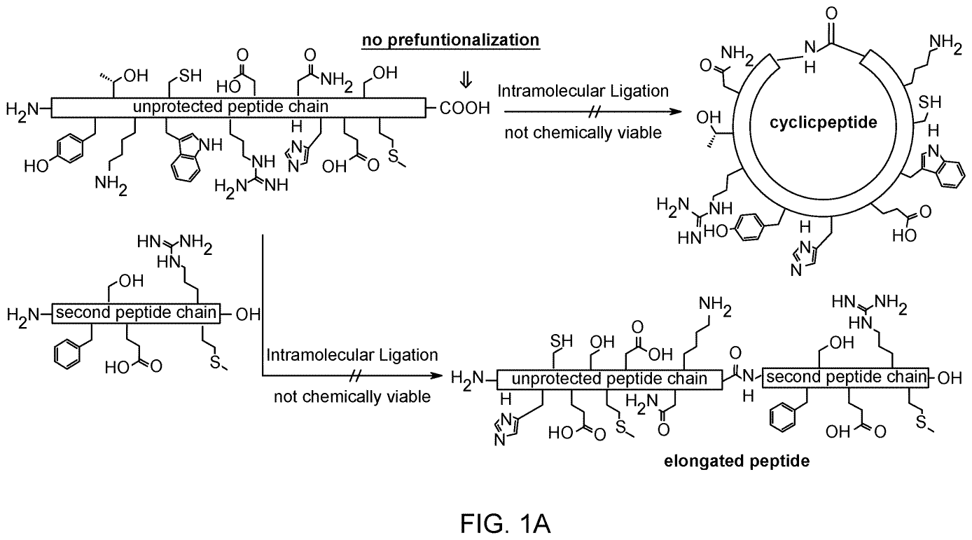

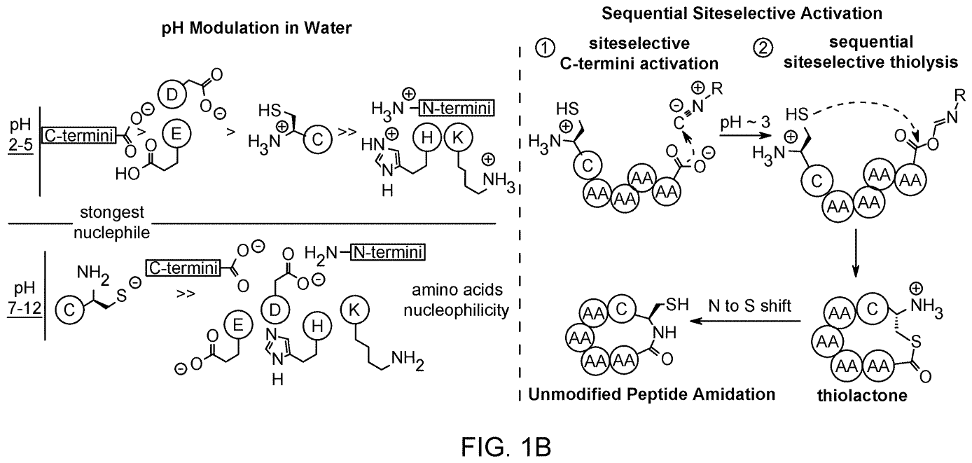

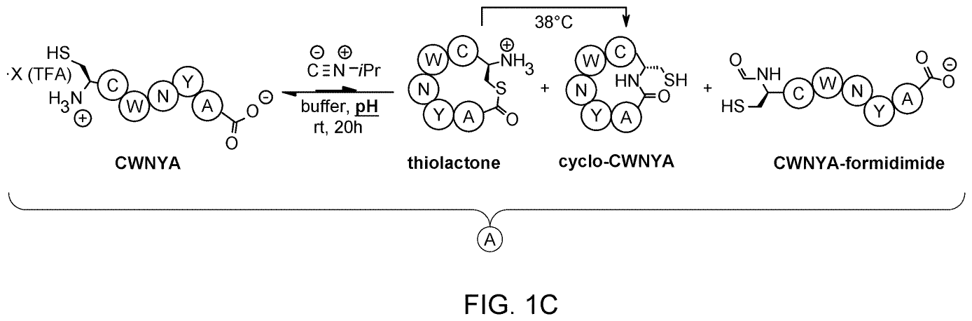

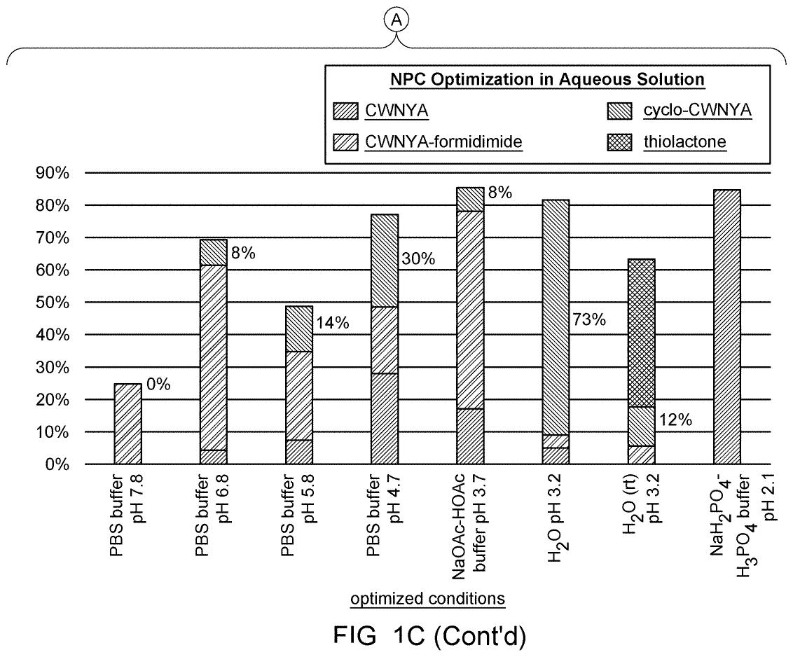

Most peptidyl sidechains and termini are nucleophilic, chemical differentiation may occur when the most nucleophilic moiety selectively engages with an electrophile (activator) among all proteinogenic residues. In Native Peptide Cyclization (NPC), crucial selectivity relies on designating the C-terminal as the primary nucleophilic residue. Note pH-dependent zwitterionic states impact residue nucleophilicity, NPC adjusts reaction pH to fine-tune sidechain and termini nucleophilicities, enabling preferential activation of C-terminal and N-terminal for eventual linear peptide cyclization. At pH levels in the near-neutral to high (7-12), cysteine thiolate ions are most nucleophilic than other residues (by 10,000-fold at pH 10-12). Meanwhile, amino sidechains, the N-terminus, and C-terminal carboxylate ions exhibit comparable levels of nucleophilicity, making them impossible to distinguish (FIG. 1b). In contrast, at low pH levels (2-5), most residues become protonated based on their pKa and isoelectric point values, and the nucleophilicities of cysteine thiol (—SH) and protonated amines (NH₃⁺) decrease significantly compared to the C-carboxylate ion (COO⁻/COOH equilibrium). Consequently, at low pH (2-5), the carboxylate ion emerges as the most nucleophilic component in a zwitterionic peptide, closely followed by thiol moieties. Thus, these reorganized nucleophilicity can be exploited for chemoselective reactions.

FIG. 1b (i.e., sequential site selective activation), presents an unprotected peptide with an N-terminal cysteine in a pH-3 buffer leads to the C-terminal (COO⁻), as the peptide's most nucleophilic entity. The reactive C-terminus preferentially combines with an electrophilic activator (isonitrile) to form a mixed-anhydride adduct, thereby achieving the C-terminal activation. The activated C-terminal is subsequently captured by the N-terminal cysteine thiol group (SH) through intramolecular thiolysis, resulting in the chemoselective formation of a thiolactone. The NPC concludes with a kinetically controlled N-to-S shift, resulting in the formation of a stable peptide amide bond, achieving cyclization.

The pentapeptide Cys-Trp-Asn-Tyr-Ala (CWNYA trifluoroacetic acid, TFA salt) was selected for the initial chemoselective NPC investigation (FIG. 1c). Treatment of the CWNYA with isopropyl isonitrile in pH 7.8 phosphate-buffer solution (PBS) at 38° C. led to exclusive N-terminal thiolation, forming CWNYA-N-formidimide via a thioformate intermediate. A decrease in the pH of the PBS solution led to the concurrent formation of the desired cyclo-CWNYA and CWNYA-N-formidimide (as shown in insert table). Notably, by decreasing the pH of the PBS solution to its lower threshold (NaH₂PO₄, pH=4.7), the yield of cyclo-CWNYA increased to 30%, indicating the clear influence of pH on the selectivity of the desired cyclization. In this regard, when lowering the pH of the solution any acid can be used, however, hydrochloric acid and trifluoroacetic acid are preferable.

Lowering the reaction pH to below 4 presented challenges. Employing several established buffer systems, such as citric acid/Na₂HPO₄ and NaOAc/HOAc, which contain competing carboxylic acids, resulted in complex and ineffective outcomes. No reaction was observed at pH near 2 with the NaH₂PO₄/H₃PO₄ buffer. Interestingly, dissolving the linear peptide CWNYA (TFA salt) in pure H₂O yielded an acidic solution (pH=3.2) and provided the best result (73% cyclo-CWNYA). The thiolactone motif was formed (46% yield) at ambient temperature, validating the proposed thiolysis.

Incorporating TFA or formic acid as additives in the preparatory HPLC mobile-phase provides the protonated polypeptides after isolation. Purified peptides dissolved in water create acidic solutions (pH approximately 2.9 to approximately 3.3 by our measurements), which conveniently reached the ideal reaction acidic for NPC. Water-soluble isonitriles are employed as specific promoters in carboxylate activation because of their broad applications in Passerini and Ugi reactions. Isonitriles are applied in coupling single amino acids with nonreactive sidechains and in polypeptide synthesis using fully protected residues. Although prior examples lacked demonstrated chemoselectivity, their efficacy in carboxylate activation confirms their suitability. Multiple commercially available isonitriles have proven effective in promoting NPC, with isopropyl isonitrile yielding superior results. The use of excess isonitrile was necessary to compensate for the competing solvolysis of isonitrile and maintain a high isocyanium ion concentration for activation. Little product formed with less than 4 equivalents of isonitrile or in its absence NPC at lower concentrations (1 mM) yielded similar product yields. Maintaining a pH around 3 was optimal, as pH values below 2 or above 5 reduced yields.

NPC successfully accomplishes intramolecular peptide ligation in an unaltered polypeptide. Around pH 3, the sequential activation of the C-terminal and the N-terminal enables peptide cyclization in aqueous conditions. The NPC process is simplified to incubating an HPLC-purified linear peptide with 10 equivalents of isonitrile in water (5 mM, pH~3) at 38° C. within 24 hours.

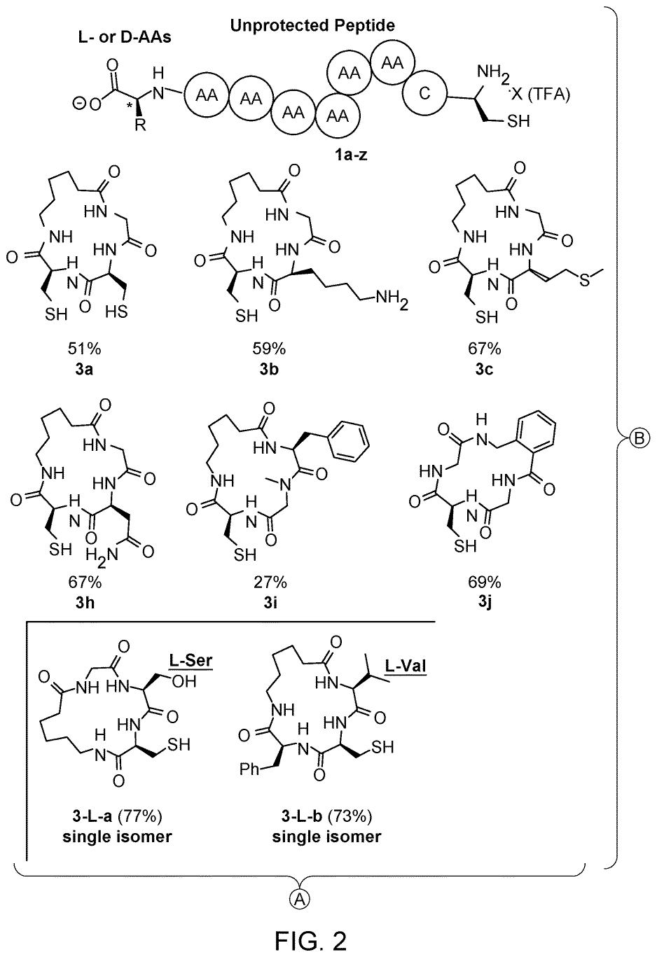

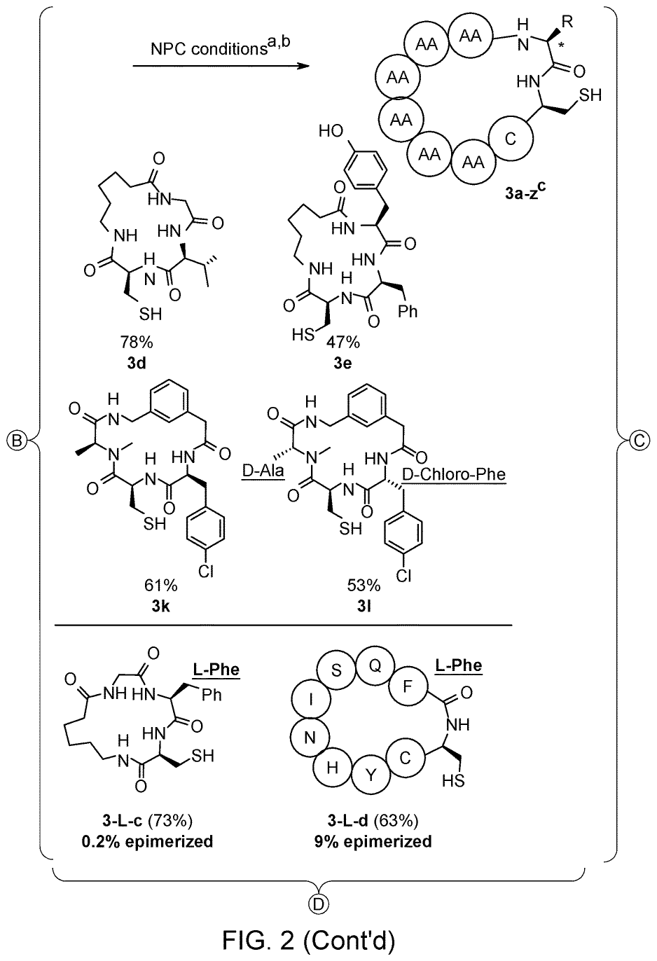

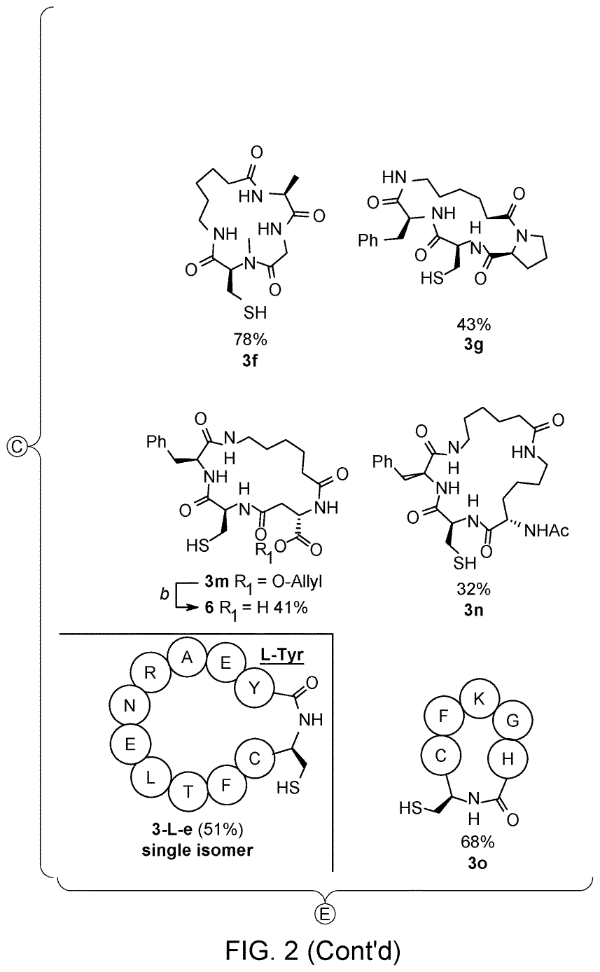

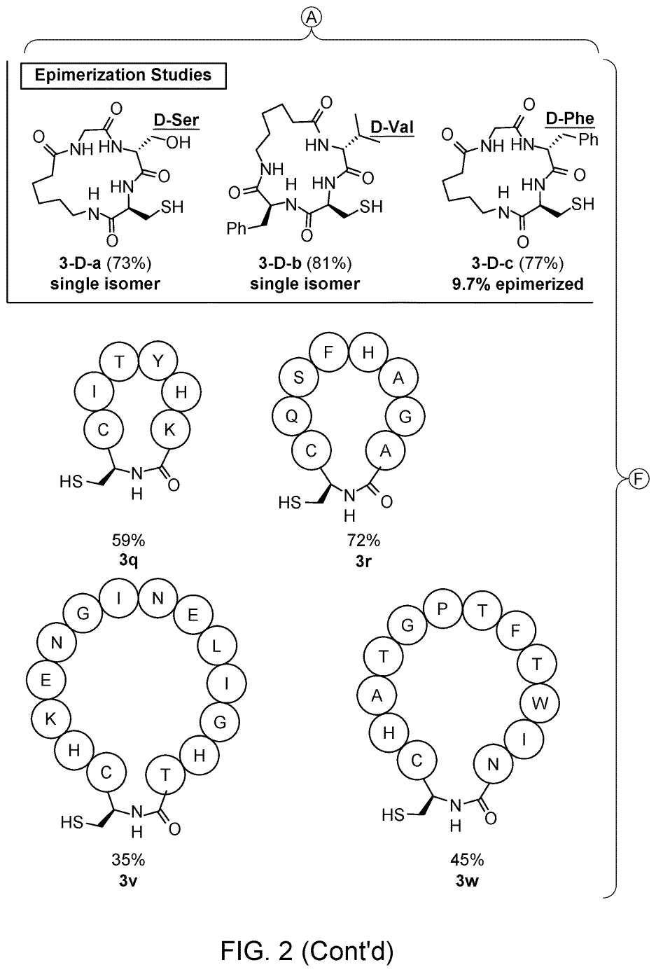

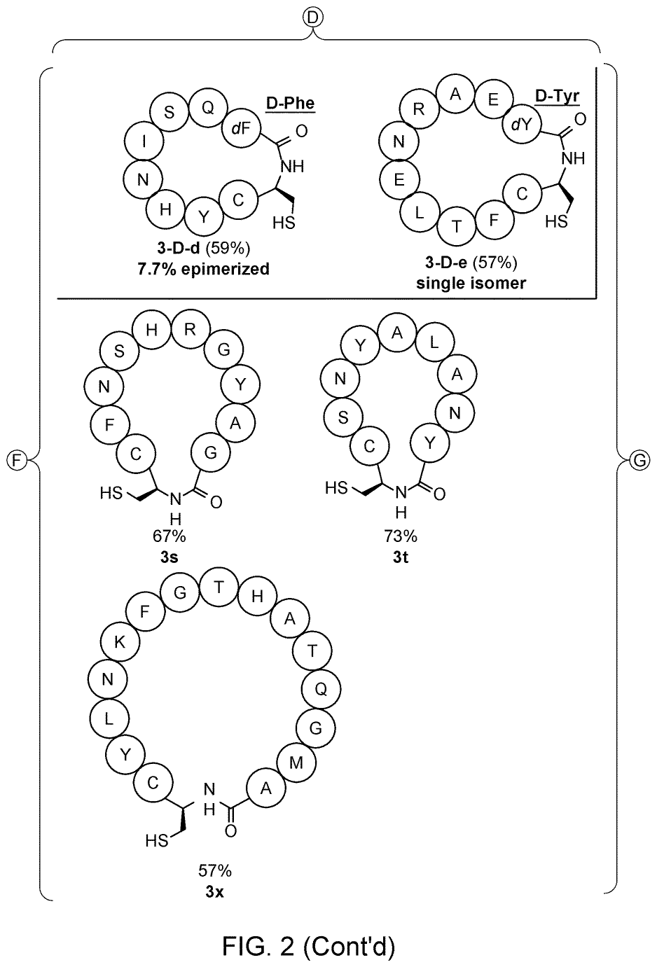

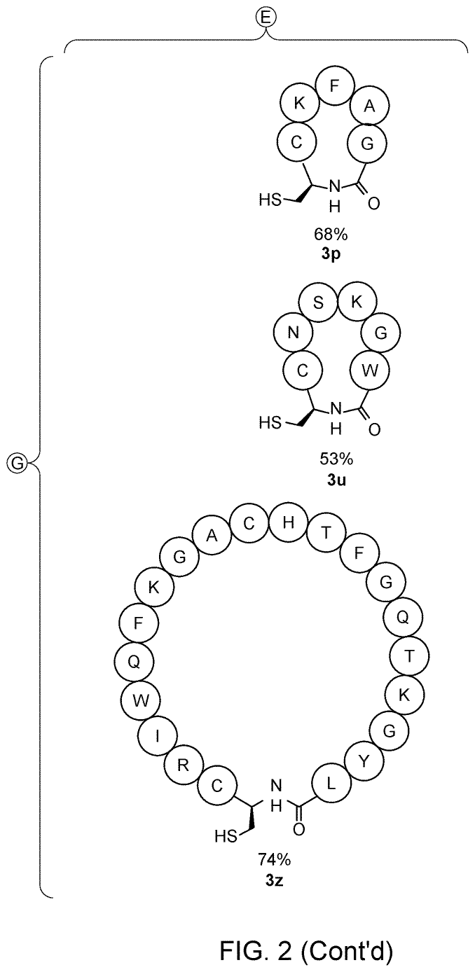

FIG. 2 presents NPC selectivity and compatibility in cyclizing 35 peptides derived from unmodified precursors. As such, a cysteine or N-methyl cysteine residue must be positioned at the N-terminus of all linear precursors, while other positions were filled with random selection of proteinogenic, D-amino acid, and noncanonical residues. The NPC method demonstrated remarkable compatibility, efficiently enabling ring closure between cysteine/N-methyl cysteine and diverse C-terminal residues. Proteinogenic tetracyclic peptides could not be formed due to constraints, while tetrapeptide cyclization with noncanonical residues (FIG. 2, see 3a-3n) was achieved. NPC effectively cyclized peptides from 5 to 20 amino acid residues, with the longest cyclic peptide of 20 residues (FIG. 2, see 3z, 74% yield), showcasing NPC's potential for larger macrocycle synthesis.

Preserving residue stereocenters is crucial for effective ligation. We assessed the potential for NPC-induced epimerization, despite no apparent racemization in the cyclized samples. Five pairs of linear peptide sequences, incorporating L- or D-amino acids known for potential C-terminal epimerization, were prepared (FIG. 2, insert). Ligation between cysteine and L-serine (3-L-a), L-valine (3-L-b), D-serine (3-D-a), D-valine (3-D-b) L-tyrosine (3-L-e) and D-tyrosine (3-D-e) furnished single isomers. However, condensation at the L- and D-phenylalanine C-termini resulted in varying degrees of epimerization, with racemization ratios ranging from 0.2% to 9.7%. Overall, these results demonstrate that the NPC process has high efficiency during ring closure with minimal epimerization.

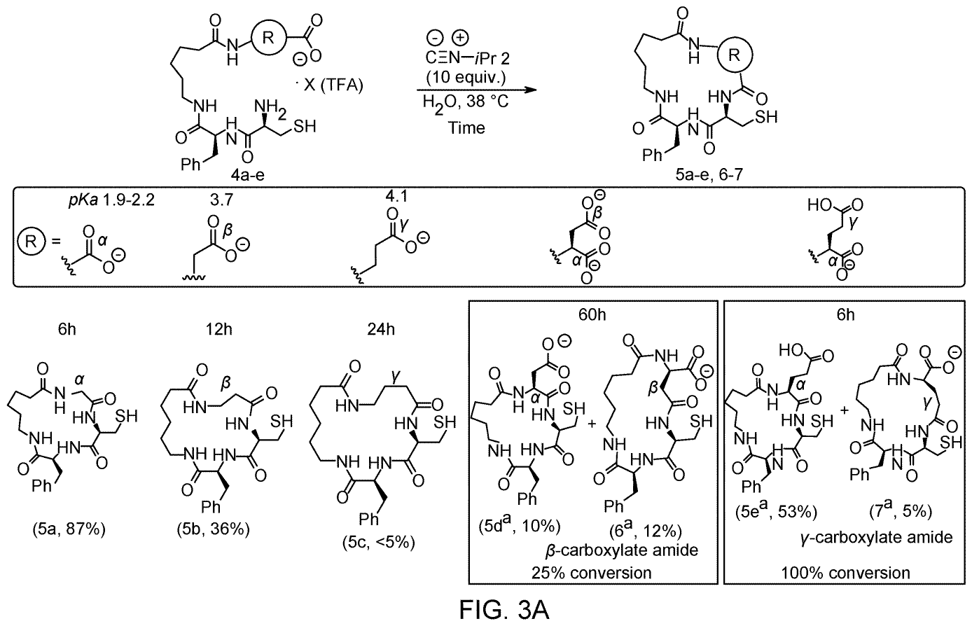

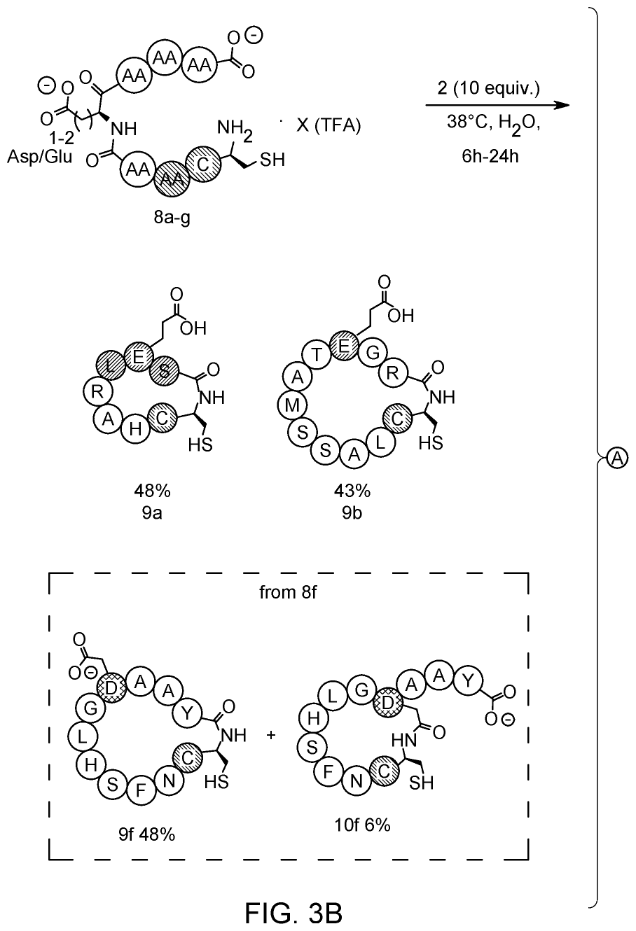

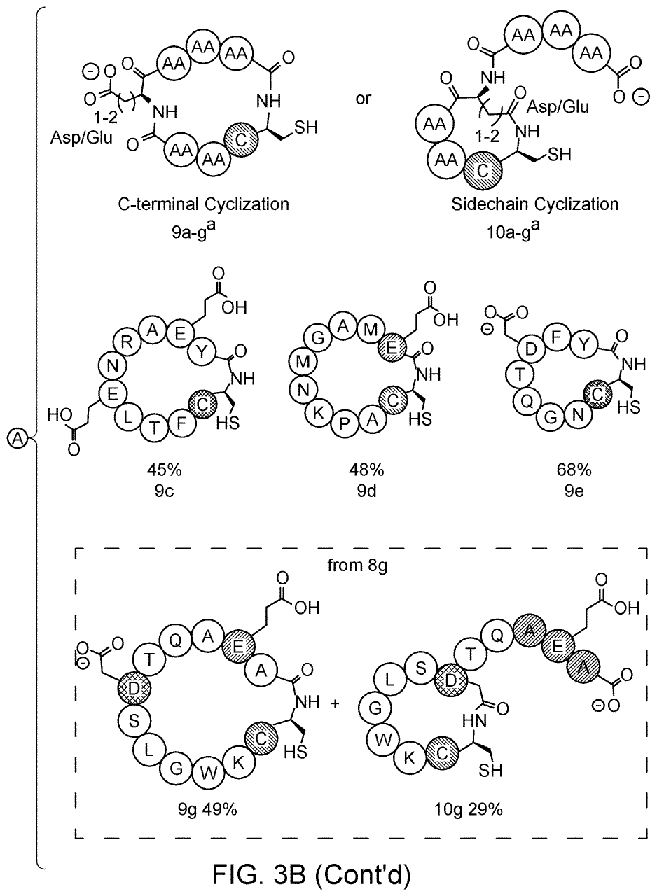

Our work differentiates selectivity among the carboxylate groups of the C-terminal and the sidechains of aspartic acid and glutamic acid. These carboxylate residues vary in terms of α-, β-, and γ-carboxylates with pKa values of 1.9-2.2, 3.7, and 4.2, respectively. Under NPC conditions (pH approximately 3), the α-carboxylate exhibits the highest proportion of deprotonated form (COO⁻) compared to the β- and γ-carboxylates, displaying prominent nucleophilicity. Hence, the C-terminal carboxylate ion is the most likely functionalized residue during initial isonitrile activation, surpassing Asp and Glu sidechain reactivity. In FIG. 3a we validate the hypothesis, five specifically designed analog sequences, Cys-Phe-Caprocid-X (X=Gly (4a), β-alanine (4b), GABA (4c), Asp (4d), and Glu (4e)), were evaluated. Cyclization of 4a produced cyclized product 5a with excellent yield (α-carboxylate, 87%, 6 h). In comparison, cyclization of 4b (β-carboxylate) required a longer time (12 h) and resulted in a lower yield (51%). Cyclization of 4c (γ-carboxylate) resulted in a poor yield (<5%) after 24 h. The disparate reaction rates and yields observed among the carboxylates under NPC conditions (α, 6 h, 87%; β, 12 h, 51%; and γ, 24 h, <5%) suggest that C-terminal carboxylate was more nucleophilic than the Asp and Glu sidechains. In competitive experiments involving both α- and γ-carboxylates within a sequence, the cyclization of Cys-Phe-Caprocid-Glu (4e) predominantly yielded the α-carboxylate cyclized product 5e (53%) within 6 hours, while γ-carboxylate condensation produced product 7 with only a 5% yield. The high selectivity of C-terminal α-carboxylate was observed in more complicated systems, a series of linear peptides (8a-8g) were subjected to NPC conditions (see, FIG. 3b), and the ratio of two possible macrocycles, C-terminal cyclization products (9a-9g) and Asp/Glu sidechain amidation moieties (10a-10g), was evaluated. Amidation of Glu-containing polypeptides (9a-9c) demonstrated consistent chemoselectivity, resulting in exclusive C-terminal condensation. The selectivity remained unchanged when Glu was positioned at the C-terminus, yielding only product 9d (40%). When compared with β-carboxylates (Asp), C-terminal cyclization was the dominant outcome observed in Asp-containing peptides, with octamer 9e displaying selective cyclization at the C-terminal α-carboxylate. Cyclopeptides 9f and 10f exhibited an 8:1 ratio of C-terminal amide and Asp sidechain amide, respectively. In peptides containing both Glu and Asp, the N-terminus preferentially reacted with alanine (9g) and the Asp sidechain (10g) in a ratio of 1.7 to 1, while the Glu sidechain remained intact. However, when the Asp was placed at the peptide C-terminus, the cyclization of Cys-Phe-Caprocid-Asp (4d) resulted in poor conversion after an extended reaction time (60 h, 25% conversion) and no clear selectivity between the α- and β-carboxylates (see. FIG. 3a). The ratio between isomers 5d and product 6 was observed to be close to 1:1.

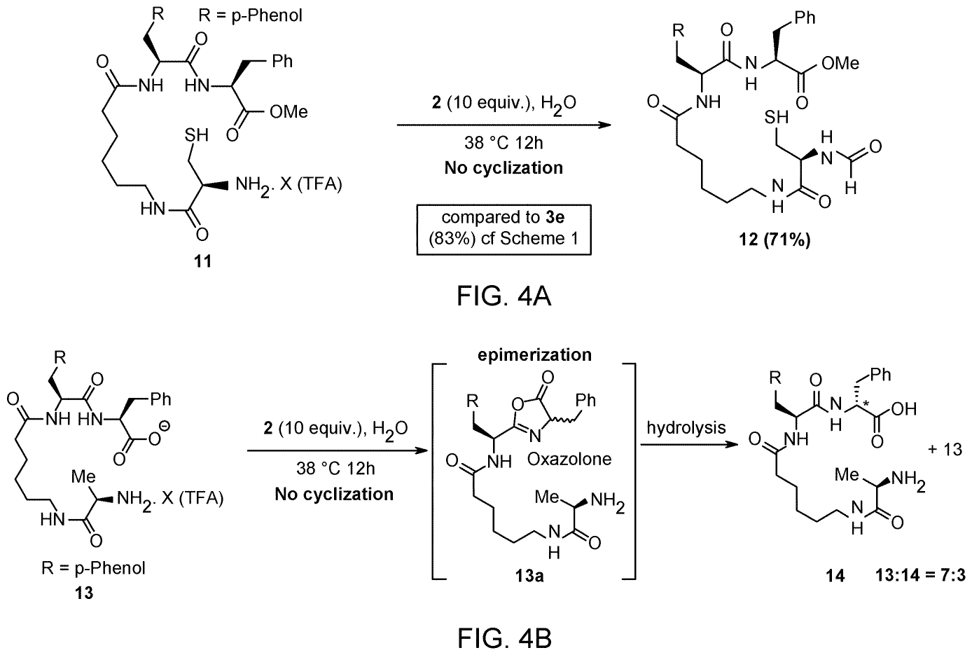

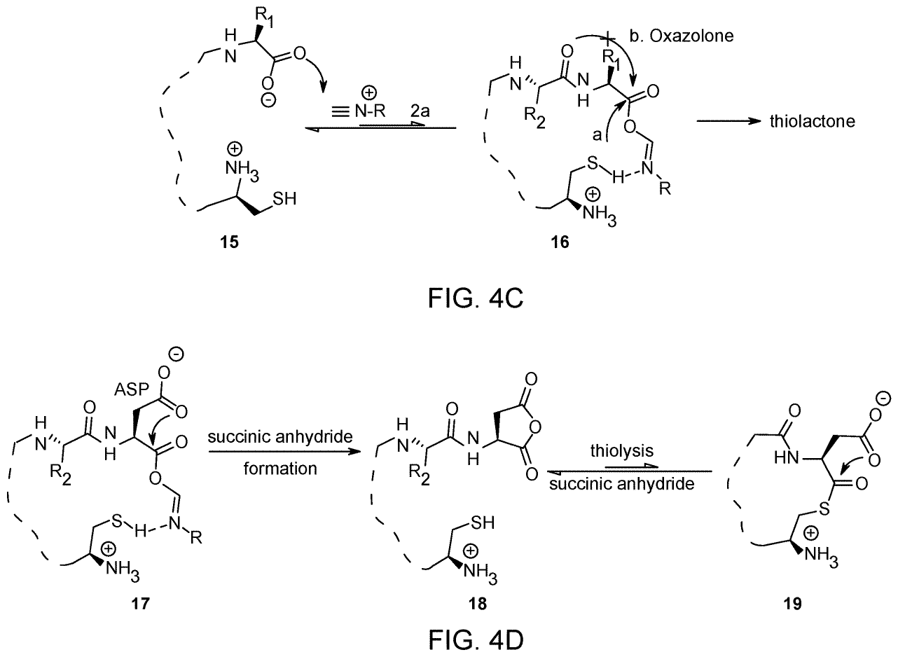

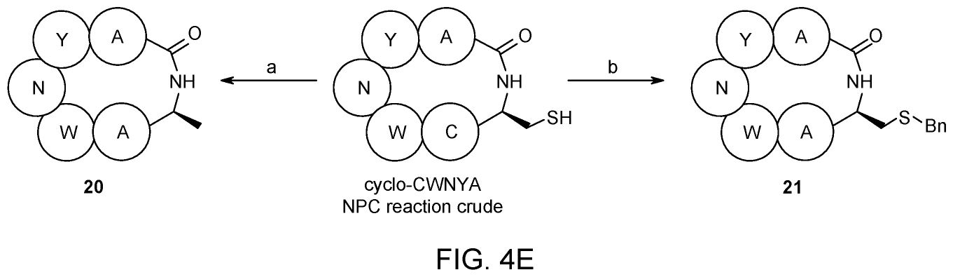

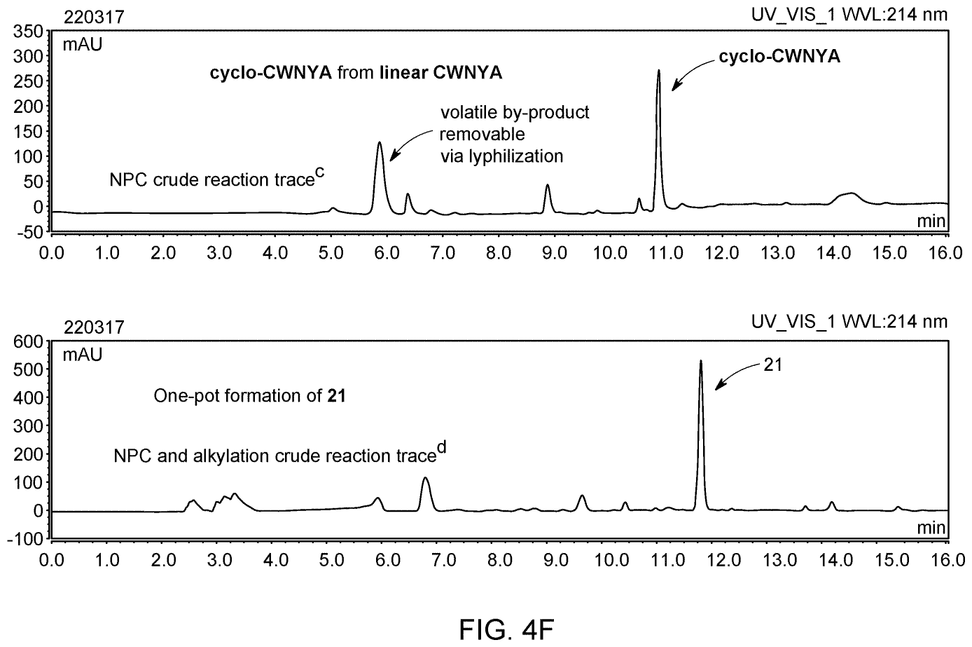

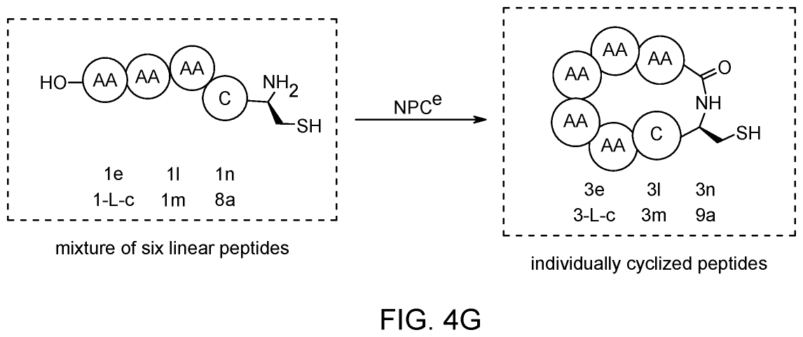

Modifying the linear peptide Cys-Caprocid-Tyr-Phe (produced 3e) at either terminus yielded peptide analogs 11 (Phe-OMe) and 13 (Cys→Ala). Both peptides failed to cyclize (FIGS. 4a, and 4b). Peptide 11 formed formamide 12 in the absence of a thiol moiety, while peptide 13 produced an epimerized isomer 14 (30%) via the oxazolone intermediate 13a (FIG. 4b). In the proposed mechanism (FIG. 4c), the combination of a carboxylate ion and a protonated isocyanium ion facilitates the formation of peptidyl formimidate carboxylate mixed anhydride 16. Although water could potentially disrupt hydrogen bonds, the intramolecular hydrogen bonding between the thiol and the imine increases the preference for thiolysis at the carboxylate carbonyl rather than the formimidate site of 16. The competing oxazolone formation (pathway b) is less favorable due to the lower nucleophilicity of the adjacent amide.

Cyclization of a C-terminal Asp residue demonstrated no selectivity and required a long reaction time of 60 h to achieve 25% conversion (cf. FIG. 3a, product 6). Perhaps due to the formation of kinetically favored succinic anhydride 18 outcompeted thiolysis (FIG. 4d). Succinic anhydride 18 underwent various equilibria, including the oscillating formation of 18 and thiolactone 19 or the hydrolysis of 18. These dynamic processes impede the cyclization and disrupt the selectivity between the α- and β-carboxylates with Asp at C-terminus. Fortunately, this issue is specific to C-terminal Asp residues and substituting Asp with either Asn or Asp-allyl ester resulted in rapid cyclization of 3h (81%, 8 h) and 3m (67%, 8 h) (cf. FIG. 2) under NPC. The inability to form a kinetically favored five-membered anhydride ring prevents the sidechains of the C-terminal Glu or mid-sequence Asp residues from competing with thiolysis. Temporary protection or strategic mid-sequence placement of Asp ensured the desired chemoselectivity, overcoming C-terminal Asp challenges.

NPC provides a clean method utilizing only volatile isonitriles, allowing for in-situ transformations (FIG. 4e). Crude cyclo-CWNYA was manipulated to cysteine-free cyclic peptide 20 via reduction or underwent in-situ S-alkylation to introduce various moieties (e.g., benzyl, 21) into the cyclic scaffold. HPLC of crude cyclo-CWNYA and 21 revealed acceptable product purities (FIG. 4f). Furthermore, the application of NPC to a mixture of linear peptides led to selective generation of six distinct macrocycles derived from their corresponding linear precursors (FIG. 4g).

### EXAMPLES

Yields refer to chromatographically and spectroscopically pure materials unless otherwise stated. All other reagents were purchased from Sigma-Aldrich, Alfa Aesar, Chemimpex and Oakwood Chemicals. All reactions were carried out in oven-dried glassware under an argon atmosphere unless otherwise noted. All commercially available materials (Aldrich®, Novabiochem®) were used without further purification. 2,2′-Azobis[2-(2-imidazolin-2-yl) propane]dihydrochloride (VA-044) was purchased from Wako Pure Chemical Industries. HATU was purchased from Genescript® (Piscataway, New Jersey). Some of linear peptides were purchased from Wuxi apptec and AlanScientific. The reaction pure Water was freshly collected from Thermo Scientific™ Barnstead™ GenPure™ Pro Water Purification System. Reactions were carried out in Fisherbrand™ Class B Clear Glass Threaded Vials.

HPLCs. Analytical High-Performance Liquid Chromatography (HPLC) and Liquid chromatography-high resolution mass spectrometry (LC-HRMS) were performed using an Agilent 1260 Infinity Quaternary LC. Purification of peptides starting materials was performed using high performance liquid chromatography (HPLC) on a Thermo Fisher Scientific Dionex Ultimate 3000 UHPLC system equipped with a UV detector and a 5 μm particle size, C-18 reverse phase HPLC column (250×10 mm). All separations involved a mobile phase of 0.5‰ TFA (v/v) in water (solvent A)/acetonitrile (solvent B) at a flow rate of 4.5 mL/min. Analytical LC-MS analyses were performed using a 5 μm particle size, C18 reverse phase HPLC column (150×2.0 mm) at a flow rate of 0.6 mL/min. LC-MS analyses were performed using a Agilent 1260 Infinity Quaternary LC with Agilent 6530 Q-TOF mass spectrometer.

Mass spectrometry: High resolution direct injection mass spectrometry by electrospray ionization (DIMS-ESI) mass spectra were recorded on Agilent 6530 Q-TOF mass spectrometer.

Nuclear magnetic resonance: 1H NMR spectra were recorded at 500 or 600 MHz at ambient temperature with Chloroform-d (CDCl3—d), Dimethyl sulfoxide-d6 (DMSO-d6) or Methanol-d4 (MeOD-d4) (Cambridge Isotope Laboratories, Inc.) as the solvent unless otherwise stated. 13C NMR spectra were recorded at 126 or 150 MHz at ambient temperature with Chloroform-d (CDCl3-d) and DMSO-d6 as the solvent unless otherwise stated. Chemical shifts are reported in parts per million relatives to CDCl3-d (1H, δ 7.26; 13C, δ 77.36) DMSO-d6 (1H, δ 2.50; 13C, δ 39.51), MeOD-d4 (1H, δ 3.34; 13C, δ 49.86). All 13C NMR spectra were recorded with complete proton decoupling.

pH Value: The reaction pH value was determined by Thermo Scientific® Orion Star A series pH benchtop meter at 25° C.

General Procedures for Peptide Synthesis: Automated Solid-Phase peptide synthesis (SPPS) was performed on a Biotage® Initiator+ Alstra™ peptide synthesis system. The Fmoc-Xxx-NovaSyn® TGT resin was employed in SPPS. Peptides were synthesized under standard automated Fmoc protocols using DMF as solvent, deblocking for 5 min in piperidine/DBU/DMF (2:2:96, V/V/V), coupling with HATU or HCTU (3.5 equiv.) and DIPEA (4.0 eqiuv.) for 25 min (‘standard cycle’), or 55 min (‘extended cycle’) for amino acids such as prolines, valines, threonines, isoleucines and arginines. The following αN-Fmoc or αN-Boc-protected amino acids from Novabiochem or Chem-Impex were employed in SPPS: Fmoc-Ala-OH, Fmoc-Arg(Pbf)-OH, Fmoc-Asn(Trt)-OH, Fmoc-Asp(OtBu)-OH, Fmoc-Glu(OtBu)-OH, Fmoc-Gln(Trt)-OH, Fmoc-Gly-OH, Fmoc-His(Trt)-OH, Fmoc-lie-OH, Fmoc-Leu-OH, Fmoc-Lys(NHBoc)-OH, Fmoc-Met-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr-OH, Fmoc-Val-OH, Boc-Glu(OtBu)-OH, Boc-Cys(StBu)-OH, Boc-Cys(Trt)-OH, Boc-Ser(OtBu)-OH, Fmoc-ε-Acp-OH, Fmoc-GABA-OH. Upon completion of the automated synthesis on a 0.04 mmol scale, the peptide resin was washed into a peptide synthesis vessel by using dichloromethane. The polypeptide was cleaved by CH2Cl2/TFE/AcOH (8:1:1, V/V/V) solution for 20 min (×3). After washing and filtration, the combined cleavage solution was concentrated under reduced pressure. The remaining residue was dissolved in a mixture of acetonitrile and water (V/V=1/1), which was lyophilized to remove the solvent. The lyophilized solid was dissolved in 2 mL TFA/TIPSH/H2O (95/2.5/2.5, V/V/V) for protecting groups removal. After acid global deprotection, the resulting solution was gently blown off by an argon stream to afford viscous liquid residue. Finally, the oily residue was washed with cold diethyl ether to yield a white solid, which was dissolved in a mixture of acetonitrile and water and ready for HPLC purification after filtration.

General Procedure for the Cyclization of Peptides Using NPC Protocol: To the unprotected peptide dissolved in pure water (c=5 mM), 10 eq of isopropyl isonitrile or tert-butyl isonitrile was added, the pH of the resultant reaction mixture was measured about 2.9 to about 3.2. The reaction mixture is left to stir for approximately 6 to 24 hours to product cyclic peptide. Extension of reaction time to 48 h if needed. The reactions involving NPC typically occur in a small vial (2-6 ml) made of hard plastic or an Eppendorf vial of the same size, with gentle stirring. To avoid the escape of volatile isonitrile, it is recommended to minimize the opening of the reaction vessel cap. If the linear peptide remained after opening the reaction vial cap, addition of extra isonitrile (5 equiv) could facilitate the cyclization of remaining linear precursor and improve the cyclization yield further. After the reaction, in the situation when cyclopeptide was formylated by the excess amount of isonitrile [M+H+28]+, 5.0 equivalents of 1M NaOH can be added to the reaction solution at room temperature for 30 min to remove S-formate. The conversion of the cyclic peptide was monitored by injecting aliquots into the Prep-HPLC and confirmed by analytical LC-MS. The NPC reaction crude solution can be lyophilized to remove unreacted isonitrile and volatile by-products prior to isolation. Alternatively, in most of the examples, the reaction solution can be directly purified by Preparatory HPLC. Preparatory HPLC was carried out with (0.5‰) Trifluoroacetic acid (TFA): water (solvent A): acetonitrile (solvent B) at detection wavelength 214 nm for product isolation. The utilization of isonitrile is interchangeable between isopropyl and t-butyl variants. Typically, isopropyl isonitrile yields 0%-15% better results in most instances.

General procedure for the cyclization of peptides using HATU condition: The fully protected linear peptide for HATU cyclization was generated by SPPS according to the general procedure supra, after the peptide was released from resin by CH₂Cl₂/TFE/AcOH (8:1:1, V/V/V). The resulting crude sequence was dissolved in acetonitrile/H2O (1:1, V/V) and lyophilized to generate light yellow fluffy solid. The resulting crude residue was dissolved in DCM/DMF (10:1, V/V) (c=2 mM). HATU (4.0 equiv.) and DIPEA (4.0 equiv.) was added into the solution. The reaction mixtures were allowed to stir at room temperature for 8h, and the solvent was removed to afford cyclization product. The crude product was dissolved in 2 mL TFA/TIPSH/H₂O (95/2.5/2.5, V/V/V) solution to remove protecting groups. After acid global deprotection, the resulting solution was gently blown off by an argon stream to afford viscous liquid. Finally, the viscous liquid was washed with cold diethyl ether to yield a white solid, which was dissolved in a mixture of acetonitrile and water and ready for HPLC purification after filtration.

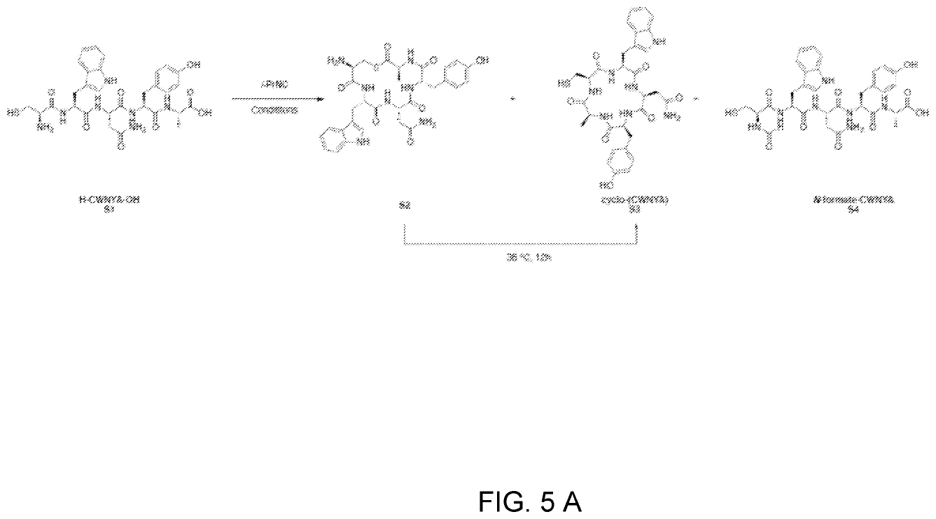

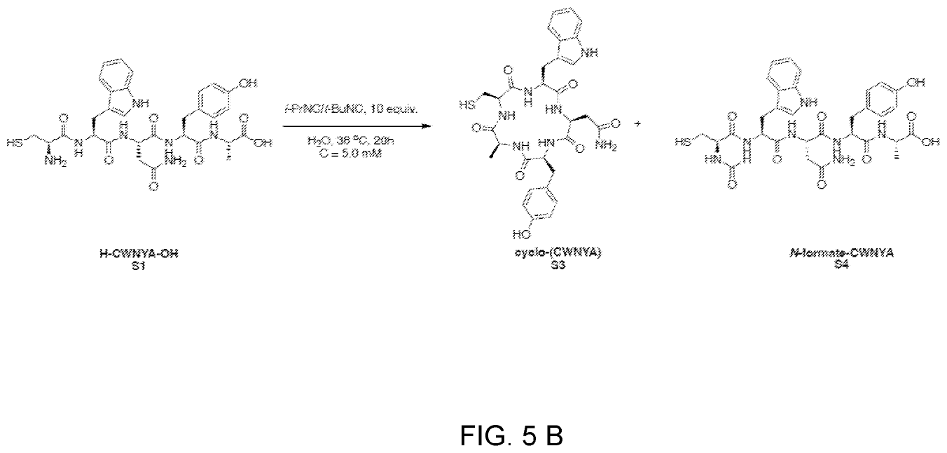

Condition Screening Experiments for Native Peptide Cyclization (NPC) and Structure Characterization: See FIG. 5A-5B for structures of H-CWNYA-OH (S1), CWNYA (S2), cyclo-CWNYA (S3), N-formate-CWNYA (S4) in connection with NPC conditions presented in Table S1.

TABLE S1
Table S1 Condition screening for NPC with H-CWNYA-OH S1

| | pH | results | | | |
|---|---|---|---|---|---|
| Conditions^{a} | value | S1 left | S4 | S3 | S2 |
| PBS buffer | 4.7 | 28% | 19% | 30% | |
| | 5.8 | 7% | 28% | 14% | |
| | 6.8 | 4% | 57% | 8% | |
| | 7.8 | 0% | 25% | 0% | |
| Citric Acid- | 2.6-4.6 | major SM left with no cyclization | | | |
| Na₂HPO₄ | | uncharacterizable by-product found | | | |
| NaOAc—HOAc | 3.7 | 17% | 61% | 8% | |
| | 4.7 | 25% | 64% | 0% | |
| Na₂HPO₄—H₃PO₄ | 2.1 | 85% | | | |
| | 2.8 | 65% | 20% | 0% | |
| H₂O | 3.0 | 5% | 4% | 73% | |
| H₂O 25 °C | 3.0 | | 6% | 12% | 46% |
| H₂O^{b} | 3.0 | 18% | 6% | 48% | |
| H₂O^{c} | 3.0 | 92% | | <5% | |
| H₂O^{d} | 3.0 | 63% | 3% | 28% | |
| H₂O^{e} | 3.0 | 43% | 4% | 48% | |
| H₂O^{f} | 3.0 | 5% | 4% | 74% | |
| H₂O^{g} | | | | trace | |
| H₂O^{h} | | | | 15% | |
| H₂Oⁱ | | | | 15% | |
| H₂O^{j} | | | | 22% | |
| H₂O^{k} | | | | 75% | |

Conditions a. All the reaction were conducted under the concentration of 5.0 mM at 38° C. for 12 h except the specifically mentioned examples; b. tert-Bu isonitrile was used in this condition; c. 2.0 eqiuv. i-PrNC was used in this condition; d. 4.0 eqiuv. i-PrNC was used in this condition; e. 8.0 eqiuv. i-PrNC was used in this condition; f. 15.0 eqiuv. i-PrNC was used in this condition; g. The reaction was conducted under the concentration of 100 mM; h. The reaction was conducted under the concentration of 50 mM; i. The reaction was conducted under the concentration of 25 mM; j. The reaction was conducted under the concentration of 10 mM; and k. The reaction was conducted under the concentration of 1 mM.

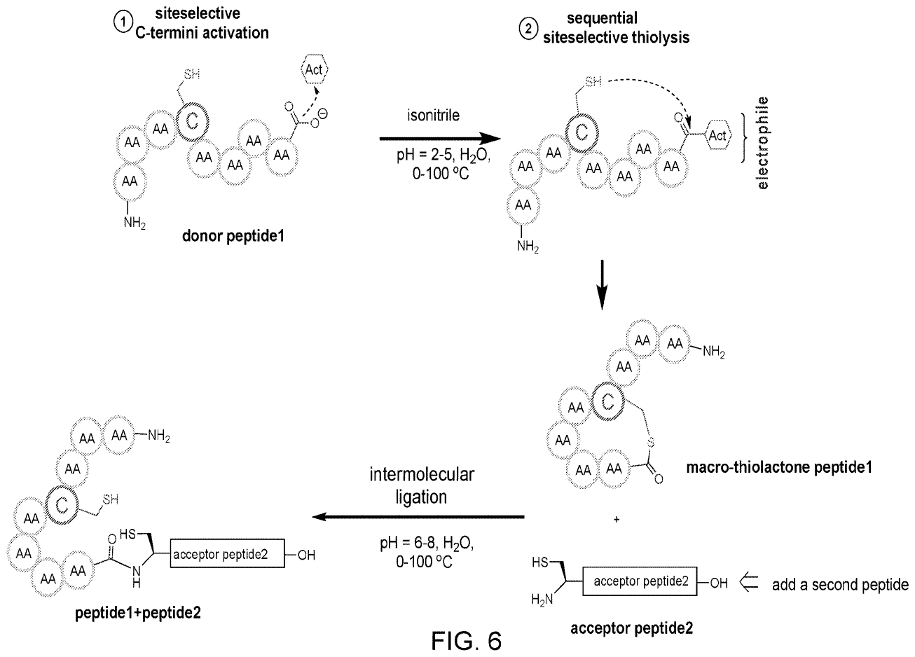

See FIG. 6 for the experimental details and structure characterization of NPC for H-CWNYA-OH (S1), cyclo-CWNYA S3, and N-formate-CWNYA S4 in connection with NPC conditions. The optimization for NPC reaction were performed based on S1 (Table S1), according to general procedure supra, different buffer conditions were carried out with either i-PrNC or t-BuNC at 38° C. Conditions employing water, i-PrNC at 5 mM concentration are identified as the optimal one and furnished the desired cyclized product in 73% yield. The NPC reaction under t-BuNC activator was slower and resulted slightly lower yield.

According to embodiments herein, the method of elongating peptides/proteins can be the elongation between a short peptide (5-40mer) and a short peptide (5-40mer). In addition, the term peptide/protein refers to the elongation between a short peptide (5-40mer) and a protein (>40mer). The method further includes the elongation between a protein (>40mer) and a short peptide (5-40mer) and between a protein (>40mer) and a protein (>40mer). As such, any residue a peptide/protein, e.g., 5-500 or more residue long sequence that is treated by the methods disclosed would be formed elongated peptides and/or proteins.

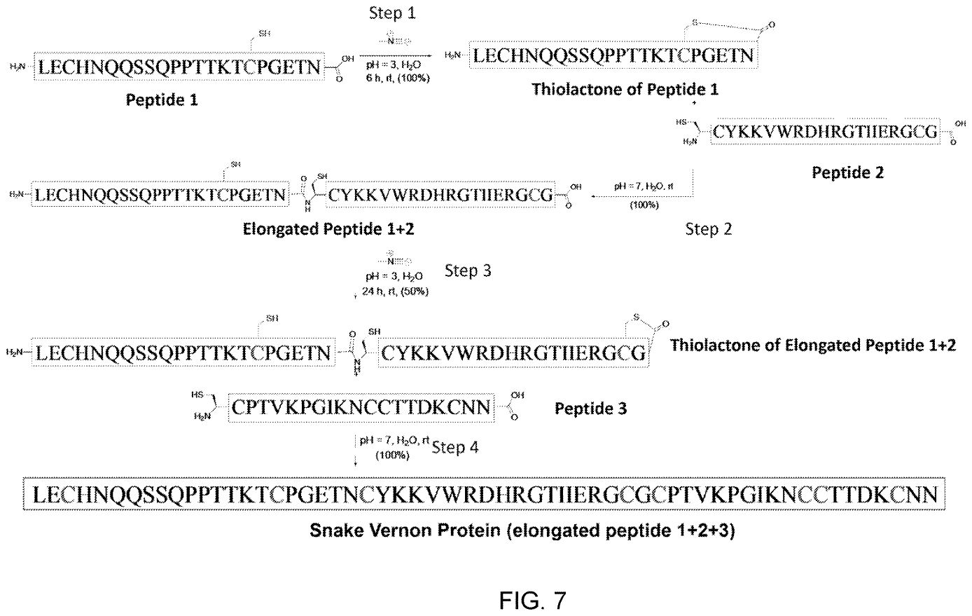

FIG. 7 illustrates the method of peptide/protein elongation and the synthesis of snake toxin protein. In this regard any residue of a peptide and/or protein, i.e., peptide/protein, for example, 5-500, or 500 plus residue long sequence is treated by an activator, e.g., isonitrile to form a macro-thiolactone first peptide. Reaction conditions remain the same as NPC conditions. While the macro-thiolactone first peptide remains in water solution, a new unprotected second peptide/protein sequence (5-500 residue long) is added to the original reaction water solution. Reaction pH is then adjusted by, e.g., Et3N or NaOH to neutral (pH=6-8) and allowed for stirring for 8-24 hours. Once either macro-thiolactone first peptide or new unprotected second peptide/protein is consumed (monitored by LCMS), an elongated peptide/protein is formed (i.e., first peptide and second peptide). Reaction is then re-adjusted back to pH=2-5 using e.g., TFA, as well e.g., isonitrile in water produced macro-thiolactone first peptide. Moreover, a third unprotected peptide/protein can be added to the original reaction mixture and adjustment of the pH to neutral is required. At ambient temperature, the first, second, and third peptide/protein (additional repeating of a third peptide/protein is contemplated) can be generated. In the FIG. 7 a protein called snake toxin was synthetically produced in one pot without workup or purification.

Thus, NPC accomplishes intramolecular and intermolecular ligation in unmodified polypeptides, marking a revolutionary breakthrough that reshapes the conventional approach to polypeptide chemical synthesis. It eliminates the need for protection, pre-modification, excessive additives, organic reagents, and solvents, greatly simplifying and expediting the cumbersome and elaborate peptide ligation process. NPC demonstrates high tolerance for proteinogenic and noncanonical amino acid residues, with a preference for amidation at the C-terminal α-carboxylate (except C-terminal Asp). Minimal epimerization was observed, and over 60 cyclopeptides were prepared. It empowers a broader range of groups expediting progress in macrocycle-based research and discovery.

### REFERENCES

Vijaya, R. P. & Jeffrey, W. B. Rethinking amide bond synthesis. Nature 480, 471-479 (2011); Che, Y. & Marshall, G. R. Engineering Cyclic Tetrapeptides Containing Chimeric Amino Acids as Preferred Reverse-Turn Scaffolds. J. Med. Chem. 49, 111-124 (2006); Thoendel, M., Kavanaugh, J., Flack, E. & Horswill, A. Peptide Signaling in the Staphylococci. Chem. Rev., 111, 117-151 (2011); Vasquez, J. & Blackwell, H. Simplified Autoinducing Peptide Mimetics with Single-Nanomolar Activity Against the Staphylococcus aureus AgrC Quorum Sensing Receptor. ACS Infect. Dis. 5, 4484-4492 (2019); Huiya, Z. & Shiyu, C. Cyclic peptide drugs approved in the last two decades (2001-2021). RSC Chem. Biol., 3, 18-31 (2022); Gause, G. F. & Brazhnikova, M. G. Gramicidin S and its use in the treatment of infected wounds. Nature 154, 703-704 (1944); Sewald, N. & Jakube, H. D. Peptides: Chemistry and Biology (Wiley-VCH, 2009); Wong, C., Lam, H., Song, T., Chen, G. & Li, X. Synthesis of Constrained Head-to-Tail Cyclic Tetrapeptides by an Imine-Induced Ring-Closing/Contraction Strategy. Angew. Chem. Int. Ed. 52, 10212-10215 (2013); Kalia, J. & Raines, R. T. Advances in Bioconjugation. Curr. Org. Chem. 14, 138-147 (2010); Silvestri, A. P., Cistrone, P. A. & Dawson, P. E. Adapting the Glaser Reaction for Bioconjugation: Robust Access to Structurally Simple, Rigid Linkers. Angew. Chem. Int. Ed. 56, 1-6 (2017); Merrifield, B. Solid phase synthesis. Science. 232, 341-347 (1986); Dawson, P. E., Muir, T. W. Clark-Lewis, I. & Kent, S. B. Synthesis of proteins by native chemical ligation. Science 266, 776-779 (1994); Ryan, H., Vishal, R. & Andrei, K. Y. Macrocyclization of Linear Peptides Enabled by Amphoteric Molecules J. Am. Chem. Soc. 132, 2889-2891 (2010); Juan, B. B. & Philip E. D. An efficient Fmoc-SPPS approach for the generation of thioester peptide precursors for use in native chemical ligation. Angew. Chem. Int. Ed. 47, 6851-6855 (2008); Zheng, J. S., Tang, S., Qi Y. K., Wang, Z. P., & Liu, L. Chemical synthesis of proteins using peptide hydrazides as thioester surrogates. Nat. Protoc. 8, 2483-95 (2013); Han, L. & Li, X. Serine/Threonine Ligation: Origin, Mechanistic Aspects, and Applications. Acc. Chem. Res. 51, 1643-1655 (2018); Jeffrey, W. B., Ryan, M. F., & Kyle, D. B. Chemoselective Amide Ligations by Decarboxylative Condensations of N-Alkylhydroxylamines and α-Ketoacids. Angew. Chem. Int. Ed. 8, 1248-1252 (2006); Pierre, C., Saidul, I. & Matthew, W. P. Peptide ligation by chemoselective aminonitrile coupling in water. Nature 571, 546-549 (2019); Frank, B. & Herbert, M. Nucleophilicities of amino acids and peptides. Org. Biomol. Chem. 5, 3814-3820 (2007); Li, X. & Danishefsky, S. J. New Chemistry with Old Functional Groups: On the Reaction of Isonitriles with Carboxylic Acids—A Route to Various Amide Types. J. Am. Chem. Soc. 130, 5446-5448 (2008); Banfi, L. & Riva, R. The Passerini Reaction. Org. React. 65, 1-140 (2005); Dömling, A. & Ugi, I. Multicomponent Reactions with Isocyanides. Angew. Chem. Int. Ed. 39, 3168-3210 (2000); Wilson, R. M., Stockdill, J. L., Wu, X., Li, X., Vadola, P. A., Park, P. K., & Danishefsky, S. J. A fascinating journey into history: exploration of the world of isonitriles en route to complex amides. Angew. Chem. Int. Ed. 12, 2834-2848 (2012); Long, F. W., Zi, W. L. & John, D. S. pH-Dependent peptide bond formation by the selective coupling of α-amino acids in water. Chem. Commun. 57, 73-76 (2021); Amadeo, F., Hans, I. & Manfred, G. The synthesis of cyclic peptides by the four component condensation (4 CC). Can. J. Chem. 57, 3257 (1979); Yu, R., Xuechen, L. & Danishefsky, S. J. Thio FCMA Intermediates as Strong Acyl Donors: A General Solution to the Formation of Complex Amide Bonds. J. Am. Chem. Soc., 131, 12924-12926 (2009); Lim, Y. Y. & Stein, A. R. Acid-catalyzed Solvolysis of Isonitriles. I. Can. J. Chem. 49, 2455-2459 (2011); Peptide TFA salt sequences ranging from 5 to 20 amino acid residues were measured the pH value of 2.9-3.2 at 5 mM concentration. It suggests the buffering ability of peptides. See Christensen, H. Proteins as buffers. Ann N Y Acad Sci. 133, 34-40 (1966); Snella, B., Diemer, V. & Drobecq, H., Agouridas, V. & Melnyk, O. Native chemical ligation at serine revisited. Organic Letters, 20, 7616-7619 (2018); Hou, J., Dariush, A.,& Julius R. Jr. Reaction of Carboxylic Acids and Isonitriles in Small Spaces. J. Am. Chem. Soc. 130, 7810-7811 (2008); and Amitai, G., Bareket, D., & Shmuel P. Protein splicing of inteins with atypical glutamine and aspartate C-terminal residues. J. Biol. Chem. 279, 3121-3131 (2004).

1. A method of cyclizing peptides, said method comprising: i) reacting a at least one unprotected peptide containing a plurality of amino acids in an acidic aqueous solution, said peptide having a C-terminus end and an N-terminus end; ii) activating the C-terminus of at least one amino acid with an electrophilic activator in the solution; and iii) maintaining the solution at a temperature of about 0° C. to about 99° C. to form at least one stable amide bond that provides an N-to-S shift and at least one cyclized peptide.

2. The method of claim 1, wherein the N-terminal amino acid contains a thiol group.

3. The method of claim 1, wherein the N-terminal is a cysteine or selenocysteine amino acid.

4. The method of claim 1, wherein the acidic aqueous solution contains hydrochloric acid, trifluoroacetic acid, or acetic acid to provide a pH of about 2 to about 5.

5. The method of claim 1, wherein the at least one peptide is a linear peptide.

6. The method of claim 1, wherein electrophilic activator is at least one water-soluble isonitrile.

7. The method of claim 6, wherein the isonitrile is at least one selected from the group consisting of isopropyl isonitrile, tert-butyl isonitrile, acetonitrile, and methyl isonitrile.

8. The method of claim 1, wherein the peptide is an HPLC-purified linear peptide.

9. The method of claim 1, wherein the solution has a pH of approximately 3 and contains approximately 10 equivalents of isonitrile (5 mM), at a temperature of about 38° C.

10. The method of claim 1, wherein the cyclized peptide contains from about 5 to about 20 amino acid residues.

12-26. (canceled)

11. The method of claim 1, wherein the peptide is obtained from chemical solid phase synthesis or protein expression.

27. A method of elongating peptides/proteins, said method comprising:
i) reacting at least one unprotected first peptide containing a plurality of amino acids in an acidic aqueous solution having a pH of about 2 to about 5 at a temperature from about 0° C. to about 99° C., said first peptide having a C-terminus end and an N-terminus end and at least one thiol containing amino acid that is not the C-terminus or the N-terminus;
ii) activating the C-terminus carboxylic acid of at least one amino acid of the first peptide with an electrophilic activator in the solution to provide thiolysis of the least one thiol containing amino acid to form at least one macro-thiolactone first peptide;
iii) adding a at least one unprotected second peptides to the solution, said second peptide having a C-terminus end and an N-terminus end and at least one C-terminus containing a thiol group and at least one amino acid containing a thiol group that is not the C-terminus; and
iv) increasing the pH of the acidic aqueous solution to about 6 to about 8 to provide for ligation of the at least one macro-thiolactone first peptide and at least one second peptide to form at least one elongated peptide.

28. The method of claim 27, further comprising the steps of reducing the solution comprising at least one elongated peptide to a pH of about 2 to about 5; adding an unprotected third peptide, said third peptide having a C-terminus end and an N-terminus end and at least one C-terminus containing a thiol group and at least one thiol containing amino acid that is not the C-terminus; adjusting the solution pH to about 6 to about 8 and temperature to about 25° C. to provide for ligation of the elongated peptide and third peptide to produce an elongated protein.

29. The method of claims 27 and 28, wherein the amino acid containing a thiol group is a cysteine or selenocysteine amino acid.

30. The method of claim 27, wherein the solution is acidified with at least one acid selected from the group consisting of hydrochloric acid, trifluoroacetic acid, or acetic acid.

31. The method of claim 27, wherein electrophilic activator is at least one water-soluble isonitrile.

32. The method of claim 27, wherein the isonitrile is at least one selected from the group consisting of isopropyl isonitrile, tert-butyl isonitrile, acetonitrile, and methyl isonitrile.

33. The method of claim 27, wherein the first and second peptides are HPLC-purified linear peptides.

34. The method of claims 27 and 28, wherein the unprotected first, second, and third peptides are obtained from chemical solid phase synthesis or protein expression.

35. The method of claims 27 and 28, wherein the unprotected first, second, and third peptides are peptide HPLC-purified linear peptide.
